SEQUENCE LISTING (12 sequences)

SEQ ID NO: 1 - DNA, 26 bp, Homo sapiens
tgaaggtcggagtcaacggatttggt

SEQ ID NO: 2 - DNA, 24 bp, Homo sapiens
catgtgggccatgaggtccaccac

SEQ ID NO: 3 - DNA, 25 bp, Homo sapiens
cgrgaagctggagaaggagaagctg

SEQ ID NO: 4 - DNA, 25 bp, Homo sapiens
caagggccgcagcttacacatgttc

SEQ ID NO: 5 - DNA, 25 bp, Homo sapiens
cctcctccatggatctgcttattca

SEQ ID NO: 6 - DNA, 26 bp, Homo sapiens
caggtcttcacctgtttgtagctgag

SEQ ID NO: 7 - DNA, 23 bp, Homo sapiens
gcgtacgcaaattaaagtccaga

SEQ ID NO: 8 - DNA, 25 bp, Homo sapiens
cagcatcctaaacagctcgcagaat

SEQ ID NO: 9 - DNA, 21 bp, Homo sapiens
ggaagaactaccagaaacgcg

SEQ ID NO: 10 - DNA, 21 bp, Homo sapiens
agatgatcagccagaggaaaa

SEQ ID NO: 11 - DNA, 26 bp, Homo sapiens
aagaggacaagaaggactaaaaatat

SEQ ID NO: 12 - DNA, 25 bp, Homo sapiens
gtagagatccagcataaagagaggt

(19) United States
(12) Patent Application Publication
(10) Pub. No.: US 2005/0186672 A1
(43) Pub. Date: Aug. 25, 2005

(54) Tissue system with undifferentiated stem cells derived from corneal limbus

(75) Inventors: Totey Satish Mahadeorao, Wadala (IN); Kashyap Subhadra Devi, Vashi (IN)

Correspondence Address: VINSON & ELKINS, L.L.P., 1001 FANNIN STREET, 2300 FIRST CITY TOWER, HOUSTON, TX, 77002-6760, US

(73) Assignee: Reliance Life Sciences Pvt. Ltd.

(21) Appl. No.: 11/043,019

(22) Filed: Jan. 25, 2005

(30) Foreign Application Priority Data
Jan. 27, 2004 (IN) 74/MUM/04

(51) Int. Cl. C12N 5/08

(52) U.S. Cl. 435/368

(57) ABSTRACT

The present disclosure describes a tissue system with self-regenerating limbal stem cells, wherein the limbal stem cells are primarily undifferentiated stem cells (USCs). The tissue system is derived from isolated corneal limbal tissue, and is suitable for restoring ocular surface impairments, particularly those that result from limbal stem cell deficiencies. The tissue system is generated by selectively augmenting the tissue system for USCs, for example by selecting and sorting cells that express stem cell-specific surface markers, such as stage specific embryonic antigen marker 4 (SSEA-4). After isolation, the USCs are cultured on a tissue base in the presence of enriched medium to generate the tissue system, which is suitable for transplantation, implantation, or grafting.

### STATEMENT REGARDING FEDERALLY SPONSORED RESEARCH OR DEVELOPMENT

Not applicable.

### BACKGROUND OF THE INVENTION

1. Field of the Invention

The present disclosure relates to tissue systems comprising mammalian undifferentiated stem cells, preferably human undifferentiated stem cells, which are derived from corneal limbus tissue and suitable for restoring damaged or diseased ocular surfaces.

2. Description of Related Art

Stem cells are responsible for cellular replacement and tissue regeneration throughout the life of an organism. Stem cells are cells that have extensive proliferation potential, and may, depending on the stem cell, differentiate into several cell lineages, and/or repopulate tissue upon transplantation. Embryonic stem (ES) cells are quintessential stem cells with unlimited self-renewal and pluripotent potential. ES cells are derived from the inner cell mass of the blastocyst stage embryo. Adult stem cells are also specialized undifferentiated stem cells, which after birth and throughout adulthood retain the ability to replace cells and regenerate tissues in an organism. It is generally understood that adult stem cells, as compared to ES cells, have less self-renewal ability and, although they may differentiate into multiple lineages, are not generally described as pluripotent. Adult stem cells (also referred to as “tissue-specific stem cells”) have been found in very small numbers in various tissues of the adult body, including bone marrow, (Weissman, (2000) Science 287:1442-1446), neural tissue (Gage, (2000) Science 287:1433-1438), gastrointestinal tissue (Potten, (1998) Phil. Trans. R. Soc. Lond. B. 353:821-830), epidermal tissue (Watt, (1997) Phil. Trans. R. Soc. Lond. B. 353:831), hepatic tissue (Alison and Sarraf, (1998) J. Hepatol. 29:678-683), and mesenchymal tissue (Pittenger et al., (1999) Science 284:143-147). In particular, adult stem cells found in the corneoscleral limbus of the mammalian eye are essential for the maintenance of a healthy ocular surface, and participate in the dynamic equilibrium of healthy ocular and corneal surfaces.

The ocular surface consists of corneal epithelium, conjuctival epithelium, and pre-corneal tear film. In a healthy eye, corneal epithelial cells are continuously shed into the tear pool and replenished by comeoscleral limbal stem cells. This renewal occurs when new cells produced by corneal limbus move centripetally from the limbus and anteriorly from the basal layer of the epithilium to replenish the corneal epithelial cells (Cotsarelis et al., (1989) Cell 57:201-209). Without proper replenishment, an unhealthy or abnormal ocular surface can result in symptoms such as unclear vision or ocular discomfort. Deficiency and depletion of limbal stem cells can result in an abnormal corneal surface, for example from the ingrowth of conjunctival elements onto the surface of the cornea, which can lead to corneal blindness, pain, photophobia, and the like (Anderson et al., (2001) Br. J. Opthalmol. 85:567-575). Loss of vision due to an abnormal corneal surface may occur despite the fact that the reminder of the eye is healthy.

Primary limbal stem cell deficiencies can be caused by hereditary conditions such as aniridia, multiple-endocrine-deficiency-associated keratitis, limbitis, and idiopathy. Aniridia is a genetically determined ocular abnormality caused by incomplete differentiation of the corneoscleral limbus, and characterized by ocular surface abnormalities, as well as absence of an iris. Secondary limbal stem cell loss may also occur from acquired conditions such as Steven-Johnson syndrome, infections (such as severe microbial keratitis), ocular surface tumors, traumatic destruction of limbal stem cells caused by chemical or thermal injury or exposure to ultraviolet radiation, multiple surgeries or cryotherapies, corneal intraepithelial neoplasia, peripheral ulcerative and inflammatory keratitis, ischemic keratitis, keratopathy, toxic effects induced by contact lens or lens cleaning fluids, immunological conditions, ocular cicatrical pemphigoid, pterygium, pseudopterygium, and the like.

Thus limbal stem cells, with their high proliferative capacity, are clearly crucial for the maintenance of a viable and healthy ocular surface, because they provide an unbroken supply of corneal epithelial cells necessary to maintain the equilibrium of the corneal surface (Tseng, (1996) Mol. Biol. Rep. 23:47-58). A depletion of limbal stem cells in a damaged or diseased eye cannot be normalized without the reintroduction of a source of limbal stem cells (Holland et al., (1996) Trans. Am. Ophthalmol. Soc. 94:677-743; Tan et al., (1996) Ophthalmol. 103:29-36). Therefore, damage due to the loss of limbal stem cells cannot be repaired without the re-introduction of a source of limbal stem cells to the eye (Tseng et al., (1996); Tsai et al., (2000) N. Engl. J. Med. 343:86-93; Henderson et al., (2001) Br. J. Ophthalmol. 85:604-609).

Several approaches have been used to attempt to restore normal vision after corneal surface impairments, however these approaches are generally not sufficient to repair damage related to or resulting from the loss of limbal stem cells. One conventional approach is to repair a damaged corneal surface due to limbal stem cell deficiency by transplanting amniotic membrane directly onto the surface of the subject's eye. (Anderson et al., (2001) Br J. Opthalmol. 85:567-575). Amniotic membrane transplantation has been found to facilitate epithelization, maintain a normal epithelial phenotype, reduce inflammation, reduce scarring, reduce adhesion of tissue, and reduce vascularization in the eye. Amniotic membrane transplantations, however, have the disadvantage of not being uniformly successful, with the final outcome often not much different then the patient's starting point (Prabhasawat et al., (1997) Arch. Ophthalmol 115:1360-67). This technique also has had limited success in restoring a normal population of corneal limbal stem cells (Shimazaki et al., (1997) Ophthalmology 104:2068-2076; Tseng et al., (1997) Am. J. Ophthalmol. 124:765-774). In addition, amniotic membrane transplantation is generally only applied in cases where patients suffer from partial limbal stem cells deficiency, since the presence of a significant population of limbal stem cells in the patient's eye enhances the likelihood of success. Methods for isolating human amniotic epithelial cells and differentiating them into corneal surface epithelium, such as disclosed by Hu et al (WO 00/73421), also have the disadvantage that they are very labor intensive and the yield of limbal stem cell is low.

Another approach to treating limbal stem cell deficiency involves corneal transplantation, which is also problematic for the treatment of chronic ocular surfaces because the ultimate success of the therapy is dependent on the gradual replacement of the donor's corneal epithelium with the recipient's corneal epithelium, which may have a poor prognosis due to a deficiency of limbal stem cells in the recipient's eye. (Lindstrom, (1986) N. Engl. J. Med. 315:57-59). Still another approach to treating limbal stem cell deficiency is to transplant limbal grafts from a donor eye into a recipient eye. This technique involves transplanting two large free conjuctival limbal grafts, each spanning approximately 6-7 mm in limbal arc length, which are harvested from a healthy eye, preferably from the same patient. This procedure has been shown in a rabbit model to restore the corneal surface more effectively than conjunctival transplantation (Tsai et al., (1990) Ophthalmology 97:446-455), and has been used to relieve ocular discomfort experienced by many patients, as well as to restore the corneal surface and vision. However, one major concern associated with this procedure is that it requires removal of large amounts of limbal stem cells from the patient's healthy eye, which may put the healthy eye at risk for a limbal stem cells deficiency, as well as other complications that may arise out of such a severe loss of limbal stem cells in the otherwise healthy eye.

Given the drawbacks of removing large limbal biopsies from a living donor, other methods have been developed for treating limbal stem cell deficiencies which rely on taking only a small biopsy of limbal epithelium from the healthy eye (Pellegrini et al., (1997) Lancet 349:990-993). These methods involve culturing the limbal biopsies on plastic petri plates for two to three weeks. Once the limbal cells are confluent, they are collected from cell culture by trypsinization and transplanted onto the patient's damaged eye. Attempts have also been made to grow the limbal biopsies on amniotic membrane (Koizumi et al., (2000) Invest. Ophthalmol. Vis. Sci. 41:2506-2513; Koizumi et al., (2000) Cornea 19:65-71; Dua et al., (2000) Surv. Ophthalmol. 44:415-425), however, limbal cells isolated and transplanted from these cultures were unstable in long term follow up, and failed to show satisfactory ocular surface repair, particularly in patients with severe limbal stem cell deficiencies (Jun Shimazaki et al., (2002) Opthalmol. 109:1285-1290). Another approach utilizing amniotic membranes is disclosed in U.S. Publication No. 20030208266, which describes the transplantation of epithelial stem cells that are cultured ex vivo on specifically treated amniotic membrane. The method for generating the surgical graft does not employ any kind of isolation step for the exclusive selection of limbal stem cells, and the distribution of the stem cell layer is non-uniform throughout the graft. These characteristics of the grafts may lead to a lack of stability of the transplanted epithelial cells, particularly in patients with limbal stem cell deficiencies.

Another approach for generating grafts is set forth in EP Patent No. 0572364, which discloses the process of growing biopsies of human eye surface epithelium in vitro, with the biopsies derived from the limbus and/or perlimbus area of the eye, or the forrinx and/or conjunctiva area of the eye. After the cells are cultured, they are transplanted into the eye by means of a suitable carrier such as sterilized gauze or a semi-rigid lens. This method, however, may fail to correct damage in patients with severe limbal stem cell deficiencies because of limited supplies of limbal stem cells in the transplanted differentiated epithelial cells. In addition, in patients with severe ocular or corneal surface damage, the insertion of a contact lens is not necessarily desirable because the presence of a contact lens on an already damaged surface may cause irritation and discomfort, and may further aggravate the condition of the eye, leading to other complications. Another patent application, WO 03/030959, discloses a corneal repair device for treating corneal lesions that uses a contact lens with a modified surface for culturing limbal stem cells. The methods has several drawbacks, including the uncertainty of whether limbal stem cells will be constantly released after being seeded on the lens to help heal the damaged eye, and the contact lens is seeded with a cell populations that may have as few as 10% limbal stem cells, which may not be adequate for successful corneal repair in patients with severe limbal stem cell deficiencies.

Similarly, U.S. Publication No. 20020039788 discloses a bioengineered composite graft for the treatment of damaged or diseased corneal epithelial surfaces, wherein the composite graft comprises a multilayered epithelium of differentiated epithelial cells. Again, these grafts of differentiated epithelial cells will have limited populations of undifferentiated limbal stem cells, which are necessary for successful ocular repair in patients severely deficient in limbal stem cells. U.S. Pat. No. 6,610,538 discloses methods of reconstructing laminae of human epithelium comeae in vitro from cultures of limbal stem cells to use as grafts for patients with ocular damage. The limbal stem cells are selected by clonal analysis and the use of markers such as K19, K12, K3, and p63. While these markers are known for indicating the corneal nature of cells, they are not particularly specific for determining whether the isolated cells are undifferentiated. In addition, the clonal analysis set forth in the disclosure selects for cells that are holoclones, which belong to the basal limbal layer, and not necessarily selecting to enrich the cell population for limbal stem cells. Therefore, these grafts do not appear to be well suited for repairing ocular damage in patients with severe limbal stem cell deficiencies.

WO 03/093457 also discloses a method for the identification and isolation of stem cells from corneal tissue by means of selecting stem cells that express the membrane protein markers CD34 or CD133, both of which belong to the differentiation cluster (CD). However, these markers are specific for isolating human haematopoietic lines rather than limbal stem cells. Therefore, this method may preferentially select for blood cells that may contaminate the corneal tissue biopsy, and the undifferentiated status of cells isolated using this method was not evaluated.

The stability and success of any limbal cell transplant depends on its ability to regenerate continuously the viable limbal stem cells for repopulating the ocular surface. The transplants or grafts currently used to treat limbal stem cell deficiencies generally contain high percentages of differentiated corneal epithelial cells rather then limbal stem cells, which may be present in only limited amounts. The donor epithelium in such transplants or grafts will survive generally for only a short period of time due to the limited supply of limbal stem cells. Alternatively, the transplants may yield a clear corneal epithelium, but the lack of sufficient limbal stem cells results in abnormal epithelial surfaces and poor healing, resulting in a failure to repair the ocular surface and improve vision. Therefore, these approaches which are intended to supply limbal stem cells to an eye with a limbal stem cell deficiency have serious limitations, which may be due to the limited supply of undifferentiated limbal stem cells with self-regenerative capacity present in the transplants or grafts. Thus, it is desirable to provide transplants or grafts that will be more successful in repairing and reconstructing ocular surface impairments by providing sufficient populations of limbal stem cells with the ability to regenerate and continually supply limbal stem cells to the eye.

### BRIEF SUMMARY OF THE INVENTION

The present disclosure describes a tissue system, wherein the tissue system comprises limbal stem cells, wherein at least about 30-90% of the cells in the tissue system are undifferentiated stem cells (USCs). Preferably, the USCs comprise at least about 30%, 40%, 50%, 60%, 70%, 80%, or 90% of the limbal stem cells in the tissue system. In various embodiments, USCs express one or more stem cell marker genes selected from the group consisting of SSEA-4, SSEA-3, Oct-4, Nanog, Rex 1, Sox2, Tra-1-60, Tra-1-81, and Stem Cell Factor. In a preferred embodiment, USCs are positive for stage specific embryonic antigen marker 4 (SSEA-4). In preferred embodiments, at least about 50%, 60%, 70%, 80%, 90%, or 95% of the USCs in the tissue system are positive for SSEA-4. In other preferred embodiments, cells in the tissue system express one or more cell surface markers selected from the group consisting of K3/K12, K19, and p63. In other preferred embodiments, the tissue system is derived from comeoscleral limbus tissue. While the corneoscleral limbus tissue may be obtained from any suitable mammal, human tissue is a particularly preferred source. Preferably the tissue system is a multi-layered tissue system.

The present disclosure also provides methods of generating a tissue system, wherein the tissue system comprises undifferentiated stem cells, comprising the steps of:
(a) isolating corneal limbal tissue from a donor;
(b) culturing the corneal limbal tissue to expand corneal limbal cells in culture;
(c) isolating a population of limbal stem cells from the cultured corneal limbal cells by sorting the corneal limbal cells to select for one or more stem cell-specific surface markers, wherein the stem cell-specific surface marker is expressed by undifferentiated stem cells (USCs);
(d) culturing the isolated population of USCs to generate the tissue system.

In preferred embodiments, the limbal stem cells comprise at least about 40-50% of the cells in the tissue system, more preferably at least about 60-70% of the cells in the tissue system, most preferably at least about 80-90% of the cells in the tissue system. In other preferred embodiments, the limbal stem cells comprise USCs, most preferably human USCs. Preferably, the USCs comprise at least about 30%, 40%, 50%, 60%, 70%, 80%, or 90% of the limbal stem cells in the tissue system. In preferred embodiments, the USCs express one or more stem cell marker genes selected from the group consisting of SSEA-4, SSEA-3, Oct-4, Nanog, Rex 1, Sox2, Tra-1-60, Tra-1-81, and Stem Cell Factor. In other preferred embodiments, cells in the tissue system express one or more cell surface markers selected from the group consisting of K3/K12, K19, and p63. The corneal limbus tissue used to generate the tissue system may be isolated from any suitable mammal, with human tissue as a particularly preferred source. Preferably, the tissue system generated according to the above methods will be suitable for transplantation, implantation, or grafting to a recipient, particularly a recipient with a limbal stem cell deficiency in one or both eyes. In other preferred embodiments, the recipient and donor are the same.

In other preferred embodiments of the above methods for generating a tissue system, the corneal limbal tissue is preferably cultured in culture media that supports the preferred growth of limbal stem cells and USCs, for example in culture media such as DMEM or F12, further supplemented with a nutrient serum and one or more soluble factors selected from the group consisting of dimethyl sulphoxide (DMSO), recombinant human epidermal growth factor (rhEGF), insulin, sodium selenite, transferrin, hydrocortisone, basic fibroblast growth factor (bFGF), and leukemia inhibitory factor (LIF). Preferably, the corneal limbal tissue is cultured until the corneal limbal cells in the culture become nearly confluent, for example 80% confluent.

In preferred embodiments, the corneal limbal tissue is cultured on an appropriate support material such as an extracellular matrix or biocoated surface, for example extracellular matrix carrier or biocoated petri dishes. Preferably, the extracellular matrix is human amniotic membrane. The support material may be biocoated with one or more attachment factors, such as fibrinogen, laminin, collagen IV, tenascin, fibronectin, collagen, bovine pituitary extract, EGF, hepatocyte growth factor, keratinocyte growth factor, hydrocortisone, or a combination thereof. The corneal limbal cells cultured on an extracellular matrix carrier are preferably dissociated from the support material prior to isolating the USCs. In preferred embodiments, the corneal limbal cells are sorted using methods well known to those of skill in the art, for example magnetic-affinity cell sorting (MACS) or fluorescence-activated cell sorting (FACS), to isolate a population of USCs. In other embodiments, one or more stem cell-specific surface markers selected to isolate USCs, including but are not limited to SSEA-4, SSEA-3, Oct-4, Nanog, Rex 1, Sox2, Tra-1-60, Tra-1-81, Stem Cell Factor, CD73, CD105, CD31, CD54, and CD117. In a particularly preferred embodiment, the stem cell-specific surface marker used to select USCs is SSEA-4. USCs may express one or more of these markers, and therefore will be preferentially selected from the population of corneal limbal cells using these markers. In certain embodiments, the sorted USCs comprise at least about 30%, 40%, 50%, 60%, 70%, 80%, 90%, or 95% SSEA-4 positive cells.

In preferred embodiments, the isolated population of USCs are cultured on a tissue base to generate the tissue system. In preferred embodiments, the tissue base is mammalian amniotic membrane, Matrigel™, laminin, collagen IV or collagen IV sheet, tenascin, fibrinogen, fibronectin, and fibrinogen and thrombin sheet (Fibrin Sealant, Reliseal™), or any combinations thereof. The tissue base may also be biocoated with a support material, including but not limited to human amniotic membrane, laminin, collagen IV, tenascin, fibrinogen, thrombin, fibronectin, or combinations thereof. In certain preferred embodiments, the tissue base is human amniotic membrane, more preferably human amniotic membrane biocoated with a support material. The isolated population of USCs is preferably cultured in medium that will allow the cells to expand without substantially differentiating, for example in culture medium enriched with conditioned medium obtained from inactivated human embryonic fibroblast cells, culture medium enriched with human leukemia inhibitory factor, or culture medium supplemented with one or more soluble factors selected from the group consisting of dimethyl sulphoxide, recombinant human epidermal growth factor, insulin, sodium selenite, transferrin, hydrocortisone, and basic fibroblast growth factor.

### BRIEF DESCRIPTION OF THE SEVERAL VIEWS OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIG. 1. H & E staining of a multi-layered tissue system on amniotic membrane at the termination point in culture.

FIG. 2. Flow cytometric analysis of cultured a limbal tissue biopsy sample. FIG. 2A shows unlabeled control cells. FIG. 2B shows cells treated with anti-mouse fluorescent isothiocyanate (FITC) antibody, a secondary antibody, as an FITC label control. FIG. 2C shows cells labeled with stage specific embryonic antigen marker-4 (SSEA-4),antibody (primary antibody) and with anti-mouse FITC (secondary antibody). Approximately 30% of the cells in the limbal tissue biopsy sample are positive for the SSEA-4 marker.

FIG. 3. Flow cytometric analysis of cultured a tissue system sample. FIG. 3A shows unlabeled control cells. FIG. 3B shows cells treated with anti-mouse FITC antibody, as a secondary antibody label control. FIG. 3C shows cells labeled with SSEA-4 antibody (primary antibody) and with anti-mouse FITC (secondary antibody). Approximately 74% of the cells in the tissue system sample are positive for the SSEA-4 marker.

FIG. 4. Immunofluorescence photomicrograph of a multi-layered tissue system showing positive immunofluorescence for SSEA-4.

FIG. 5. Immunofluorescence photomicrograph of a multi-layered tissue system showing positive immunofluorescence for Stem Cell Factor (SCF).

FIG. 6. Immunofluorescence photomicrograph of a multi-layered tissue system showing positive immunofluorescence for Tra-1-60.

FIG. 7. Immunofluorescence photomicrograph of a multi-layered tissue system showing positive immunofluorescence for Oct-4.

FIG. 8. Immunocytochemistry photomicrograph of a multi-layered tissue system showing positive immunofluorescence for p63. The immunoperoxidase assay was performed using the Vector Elite Kit. p63 is a nuclear antigen and the brown colored spots are the positive nuclei of the cells in the tissue system.

FIG. 9. Immunofluorescence photomicrograph of a multi-layered tissue system showing the focal presence of K3/K13 positive cells in the superficial layers of the tissue system.

FIG. 10. Immunofluorescence photomicrograph of a multi-layered tissue system showing the basal layer expression of K19 in the tissue system.

FIG. 11. Immunofluorescence photomicrograph of a multi-layered tissue system showing the absence of Connexin 43 expression in the tissue system using the Vector Elite Kit. Expression of Connexin 43 has been shown to be absent in the limbal basal epithelium.

FIG. 12. Gene expression profiling of the cell population of a multi-layered tissue system by RT-PCR of the undifferentiated stem cell markers Oct-4, Nanog, Rex1, as well as BMP2 and BMP5. GAPDH expression was also analyzed as a positive control. Expression of all cell markers tested was found.

FIG. 13. Bar graph demonstrating the viability for the present disclosure of limbal tissue biopsies in transportation medium 12, 24, 48, and 72 hours after surgical collection. Viability was measured by the percentage success rate of developing multi-layered tissue systems from the limbal tissue biopsies.

FIG. 14. Bar graph demonstrating the viability of multi-layered tissue systems at 6, 12, 24, and 48 hours post-culture. The tissue systems were transported at either at 4° C. or room temperature, which affected the viability of the tissue systems over longer periods of time. Viability was measured by the percentage success rate (survival) of cells in the tissue system.

### DETAILED DESCRIPTION OF THE INVENTION

The present disclosure describes a tissue system comprising undifferentiated stem cells (USCs) that are preferably self-regenerating and derived from corneal limbus tissue, as well as methods for generating the tissue system and applications thereof. As used herein, a “tissue system” is a population of cells comprising USCs, preferably on an appropriate tissue base, suitable for transplantation, implantation, or graft to a mammalian subject. Preferably, the tissue system is a transplant, implant, or graft, for example a surgical graft or a composite graft, with multi-layered aggregates of cells. As used herein, the term “undifferentiated stem cells” or “USCs” refers to undifferentiated or substantially undifferentiated cells or uncommitted progenitor cells, which express one or more stem cell-specific markers, preferably embryonic stem cell-specific markers. USCs of the present disclosure exhibit ES cell-like characteristics and properties such as, for example, expression of ES-specific markers, for example SSEA-4, SSEA-3, Oct-4, Nanog, Rex 1, Sox, Tra-1-60, and/or long-term proliferation in culture. In addition, USCs of the present disclosure may proliferate and generate progeny, which may be undifferentiated or differentiated cells, depending on environmental conditions. For example, USCs of the present disclosure have the potential to differentiate into corneal epithelial cells, which are essential to the healthy function of the ocular surface of the eye. As used herein, the term “differentiation” refers to a process whereby undifferentiated stem cells or precursors cells acquire a more specialized fate.

In preferred embodiments, the tissue system with USCs is derived from comeoscleral or corneal limbus tissue from a human donor. In particular, the present disclosure is a tissue system with self-regenerating USCs, and preferably comprises a large population of USCs, for example at least about 70%, at least about 80%, or at least about 90% USCs. The presence of a large population or high percentage of USCs in the tissue system greatly facilitates the ability of the tissue system to restore damaged or diseased ocular surfaces after transplantation, implantation, or graft to a mammalian subject. In addition, the high proportion of USCs in the tissue system allows the system to be stable for a longer period of time by continuously repopulating the ocular surface with viable limbal stem cells, which are essential to the healthy functioning of the ocular surface. In certain embodiments, the tissue system is a composite graft comprising an extracellular matrix carrier, for example, amniotic membrane, having a plurality of USCs, wherein the plurality of USCs are cultured ex vivo on the extracellular matrix carrier.

Preferably the tissue system is transplanted, implanted, or grafted onto a damaged or diseased eye and able to repair ocular surface impairments, particularly in subjects with severe limbal stem cell deficiencies in the damaged or diseased eye. As used herein, a subject with a severe limbal stem cell deficiency in a damaged or diseased eye may have a complete absence of limbal stem cells in the damaged or diseased eye. In preferred embodiments, the donor of the limbal tissue biopsy used to generate the tissue system with USCs is also the recipient of the tissue system transplant, implant, or graft (i.e., autologous tissue system). Alternatively, when the donor of the limbal tissue biopsy is not the recipient, the donor is preferably a bio-compatible donor, for example a close relative of the recipient of the transplant or graft, or may also be from a bio-compatible (e.g., histocompatible) cadaver (i.e., allogenic tissue system). It is generally desirable that transplanted cells or tissues be genetically identical to the recipient of the transplant in order to avoid problems with tissue rejection.

A significant advantage of using corneal limbal tissue as the source to derive a tissue system as disclosed herein is the relative ease in obtaining corneal limbal tissue from a donor. The process requires only minor surgery that is safe, simple, and efficient, and only small biopsies of corneal limbus tissue are needed. The corneal limbal tissue is found in the cornea, which is a transparent, avascular tissue that is located at the outer surface of the anterior eye. It provides protection from environmental insult, and allows for the efficient transmission of light into the eye. The cornea is comprised of two main compartments: (1) the anterior non-cornified stratified squamous epithelial layer and (2) the underlying substantia propria. The human cornea harbors three known cell types: corneal epithelial cells; stromal keratocytes (corneal fibroblast); and an underlying layer of stromal associated corneal endothelial cells. Comeal epithelium is a cellular multiplayer that is five to seven cells thick and covers the anterior surface of the cornea. Ordinarily, a natural turnover of corneal epithelial cells takes place in which superficial epithelial cells are shed from the epithelial surface and replaced by those from below. Basal epithelial cells, migrating inward from the periphery, replenish the population of deeper corneal epithelial cells.

Corneal limbus (also known as corneoscleral limbus) is an annular transitional zone approximately 1 mm wide between the cornea and the bulbar conjunctiva and sclera. It appears on the outer surface of the eyeball as a slight furrow marking the line between the clear cornea and the sclera. It is highly vascular and is involved in the metabolism of the cornea. Limbal and conjuctival epithelial cells, together with a stable pre-ocular tear film, maintain the integrity of the cornea. While it is known that the source of the replenished corneal epithelial cells are adult stem cells, the exact location and properties of these cells were unknown. The existence of a population of USCs in the corneal limbus with ES cell-like characteristics was unknown until isolated and characterized by the present inventors. The present disclosure describes a tissue system with a high percentage of USCs derived from the corneoscleral limbus, which may be used to restore the ocular surface of a damaged or diseased eye.

A typical procedure for isolating corneal limbal tissue is to surgically remove a small biopsy consisting of 0.8-3 mm² of limbal tissue from the superior or temporal quadrant of the corneal surface of the donor's eye. Procedures for obtaining such biopsies from the corneal limbus, for example by lamellar keratectomy, are known to those of skill in the art. Once a biopsy is removed from a donor, it must be transported to a facility so that the limbal tissue biopsy can be cultured into a tissue system disclosed herein. It is important that a sufficient portion of the limbal tissue biopsy remain viable during transport so that a tissue system can be derived therefrom. Preferably, the limbal tissue biopsy is transported or stored in a medium which supports the viability of the biopsy. A preferred medium for transporting the biopsy comprises of Dulbecco's Modified Eagles Medium (DMEM) and Ham's F-12 (ratio 1:1), supplemented with human cord blood serum (3-5%), DMSO (0.1-0.5%), rhEGF (0.5-2 ng/ml), insulin (0.5-5 μg/ml), transferrin (0.5-5 μg/ml), sodium selenite (0.5-5 μg/ml), hydrocortisone (0.1-0.5 μg/ml), cholera toxin A (0.01-0.1 μmol/l), gentamycin (10-50 μg/ml), and amphotericin B (0.5-1.25 μg/ml). Preferably the limbal cell biopsies are place in culture within 48 hours of surgical removal from the donor.

After limbal tissue is biopsied from a donor, it is placed in culture with culture media, preferably with an appropriate support material such as an extracellular matrix or biocoated surface, for example extracellular matrix carrier or biocoated petri dishes. The limbal tissue biopsy may either be cultured as an intact explant, or may be dissociated into a single cell suspension prior to being cultured. In preferred embodiments the presence of a support material facilitates the binding of the limbal stem cells in the biopsy to the tissue culture plate, thereby facilitating the growth of the limbal stem cells. Preferably the explant is cut into small pieces before being placed in culture. Examples of support materials useful for culturing limbal tissue include but are not limited to Matrigel™ and its equivalents, mammalian amniotic membrane, preferably human amniotic membrane, laminin, tenascin, entactin, hyaluron, fibrinogen, collagen-IV, poly-L-lysine, gelatin, poly-L-ornithin, fibronectin, platelet derived growth factor (PDGF), and the like, either alone or in combination with other support materials. The support materials may also be treated with one or more additional growth factors or attachment factors, for example fibrinogen, laminin, collagen IV, tenascin, fibronectin, collagen, bovine pituitary extract, EGF, hepatocyte growth factor, keratinocyte growth factor, hydrocortisone, or combinations thereof

Human amniotic membrane is preferred for culturing biopsied limbal tissue, and can be prepared using methods well known to those of skill in the art (see, for example, U.S. Pat. No. 6,152,142, and Tseng et al., (1997) Am. J. Ophthalmol. 124:765-774, each incorporated herein by reference). For example, amniotic membrane may be prepared to enhance the growth of limbal stem cells by removing endogenous amniotic epithelial cells by freeze-thawing, enzymatic digestion, and mechanical scraping, followed by the treatment of the surface with growth factors, extracellular matrix compounds, and/or adherence-enhancing molecules. Amniotic membrane is a preferred substrate for generating the tissue system because it is a natural substrate which facilitates the viability and growth of USCs. In one embodiment, the amniotic membrane, with the basement membrane or stromal side up, is affixed smoothly onto a culture plate for culturing USCs. Preferred methods of using extracellular matrix materials or biocoated surfaces are described in the examples below.

A preferred method of culturing the limbal tissue biopsies is to subject the explant to dry incubation for several minutes, either before or after placing the explant on an extracellular matrix or biocoated tissue culture plate. A small amount of culture medium is then added to the explant so that it sticks to the extracellular matrix or biocoated tissue culture surface. After several hours to a day, additional media is gently added and the explant is incubated for several days at 37° C. in a CO₂ incubator, changing the media every alternate day. Preferably, the pieces of the original limbal tissue biopsy are removed from the culture after stem cells begin proliferating in the culture. In other embodiments, the limbal tissue biopsy may be used to generate a single cell suspension, which is subsequently cultured to generate the tissue system disclosed herein. For example, the limbal tissue biopsy is washed and then enzymatically treated, for example with trypsin-EDTA (e.g., 0.25% for 20-30 minutes) or dispase (e.g., overnight at 4° C.), to generate a single cell suspension which includes USCs. Enzymatic treatment allows for separation of the epithelium; therefore, stroma or mesenchymal cells may be reduced or absent in the single cell suspension.

The preferred media used for culturing the limbal tissue is DMEM or DMEM:F-12 (1:1) media, preferably supplemented with a nutrient serum, for example a serum or serum-based solution that supplies nutrients effective for maintaining the growth and viability of the cells (e.g., knock-out serum, heat-inactivated human serum, or human cord blood serum). The media may also be supplemented with growth factors. As used herein, the term “growth factor” refers to proteins that bind to receptors on the cell surface with the primary result of activating cellular proliferation and differentiation. The growth factors used for culturing limbal tissue are preferably selected from epidermal growth factor (EGF), basic fibroblast growth factor (bFGF), leukemia inhibitory factor (LIF), insulin, sodium selenite, human transferrin, or human leukemia inhibitory factor (hLIF), as well as combinations thereof. However, any suitable culture media known to those of skill in the art may be used. In certain embodiments, the limbal cells are treated with cytokines or other growth factors which cause the USCs to preferably proliferate in the culture.

After the limbal tissue is cultured for several days, for example until the cells become confluent, the USCs can be isolated from the culture. Preferably, the limbal tissue culture is allowed to grow until it is at least about 50%, 60%, 70%, 80%, 90%, or 95% confluent. In preferred embodiments, the limbal cells are first dissociated from the extracellular matrix or biocoated tissue culture plate, preferably through enzymatic digestion, for example using trypsin-EDTA or dispase solutions. The USCs can be isolated from the other limbal cells in the culture using a variety of the methods known to those of skill in the art such as immunolabeling and fluorescence sorting, for example solid phase adsorption, fluorescence-activated cell sorting (FACS), magnetic-affinity cell sorting (MACS), and the like. In preferred embodiments, the USCs are isolated through sorting, for example immunofluorescence sorting of certain cell-surface markers. Two preferred methods of sorting well known to those of skill in the art are MACS and FACS.

Sorting techniques such as immunofluorescence-staining techniques involve the use of appropriate stem cell markers to separate USCs from other cells in the culture. Appropriate stem cell specific surface markers that may be used to isolate USCs from cultured limbal cells include but are not limited to SSEA-4, SSEA-3, CD73, CD105, CD31, CD54, and CD117. In preferred embodiments, USCs are isolated by MACS through the use of a cell surface marker such as SSEA-4. By this means, enriched populations of cell-surface marker positive USCs are obtained from the mixed population of cells cultured from the limbal tissue biopsy. Alternatively, the cells can be sorted to remove undesirable cells by selecting for cell-surface markers not found on USCs. In the case of USCs isolated from limbal tissue, USCs are negative for the following cell-surface markers: CD34, CD45, CD14, CD133, CD106, CD11c, CD123, and HLA-DR.

The enriched limbal cell cultures obtained by sorting have at least about 10%, 20%, 30%, 40%, 50%, 60%, 70%, 80%, 90%, 95%, 98%, or 99% USCs. In preferred embodiments the isolated cells will be at least about 50%, 70%, 80%, 90%, 95%, 98%, or 99% SSEA-4 positive USCs. In alternative embodiments, mixed cell cultures containing limbal cells are screened for the presence of USCs by screening for expression of certain gene markers. In the case of mixed limbal cell cultures, populations of USCs can be identified by the expression of gene markers such as SSEA-4, SSEA-3, OCT-4, Nanog, TDGF, UTX-1, FGF-4, Tra-1-60, Tra-1-81, stem cell factor (SCF), Sox 2, Rex 1, as well as other gene marker of undifferentiated cells, or combinations thereof.

After the population of limbal cells enriched for USCs is isolated using one of the above methods, the isolated cells are preferably cultured under conditions and in a media that supports the growth of USCs and the development of a tissue system for transplanting, implanting, or grafting onto a damaged or diseased eye. Preferably the tissue system cultured under these conditions will comprise at least about 30%, 40%, 50%, 60%, 70%, 80%, 90%, or 95% USCs. In a preferred embodiment, the isolated USCs are cultured on a tissue base in the presence of an enriched medium for developing the tissue system with USCs. Preferably, the tissue base has characteristics which approximate the natural ocular surface, for example characteristics such as being clear, thin, elastic, biocompatible, non-vascular, and non-antigenic, and can also support the growth of USCs, as well as normal differentiation after transplant, implant, or graft.

In preferred embodiments, the tissue base is selected from mammalian amniotic membrane, Matrigel™ and its equivalents, laminin, tenascin, entactin, hyaluron, fibrinogen, thrombin, collagen-IV, collagen-IV sheet, poly-L-lysine, gelatin, poly-L-ornithin, fibronectin, platelet derived growth factor (PDGF), thrombin sheet (Fibrin Sealant, Reliseal™, Reliance Life Sciences), and the like, or combinations thereof. In other embodiments, the tissue base is a collagen gel or a fibrin gel, and the gel may further comprise other desirable cell types for generating the tissue system, including but not limited to fibroblasts, such as corneal stromal fibroblasts, derivatives of mesenchymal tissue, and epithelial cells, such as corneal epithelial cells. In still other embodiments, the tissue base is a hydrogel, for example a synthetic hydrogel, a soft hydrogel contact lens or a poly-HEMA matrix. In preferred embodiments, the tissue base will be gradually resorbed in vivo after transplant, implant, or graft of the tissue system. In addition, the tissue base is preferably non-antigenic, and facilitates epithelialization without significant fibrovascular growth.

The preferred tissue base for use in generating the tissue system disclosed herein is human amniotic membrane, which may be prepared using methods well known to those of skill in the art. The human amniotic membrane may be used intact with the epithelial surface, or denuded of epithelial cells. Preferred methods for preparing the human amniotic membrane are disclosed in the Examples below. In other preferred embodiments, the tissue base is biocoated with an additional support material, for example a material that facilitates binding of USCs onto the tissue base. The additional support material that may be employed is preferably selected from fibrinogen, laminin, collagen IV, tenascin, fibronectin, collagen, bovine pituitary extract, EGF, hepatocyte growth factor, keratinocyte growth factor, hydrocortisone, or combinations thereof.

The preferred enriched media used for culturing limbal cells to generate the tissue system is DMEM or DMEM:F-12 (1:1) media, preferably supplemented with a nutrient serum, for example a serum or serum-based solution that supplies nutrients effective for maintaining the growth and viability of the USCs (e.g., knock-out serum, heat-inactivated human serum, or human cord blood serum). The media may also be supplemented with growth factors. The media may also be enriched with a conditioned medium obtained from inactivated human embryonic fibroblast culture medium (e.g., 30-50%), or culture medium supplemented with hLIF (e.g., 4-12 ng/ml) or other growth factors that facilitate growth of USCs. Other factors used for culturing limbal cells are preferably selected from DMSO, hydrocortisone, EGF, bFGF, LIF, insulin, sodium selenite, human transferrin, laminin, fibronectin, and the like, as well as combinations thereof Preferably the growth facilitating agents used to culture the limbal cells at any stage are of human recombinant origin. The enriched media may also be used to transport the tissue system prior to transplantation, implantation, or grafting.

In other preferred embodiments, the media used to prepare the tissue system, including the medium used to transport the limbal tissue biopsies, the medium used to culture the biopsies, the enriched medium used to culture the limbal stem cells, and the medium used to transport the tissue system, do not contain any sera or other factors of animal origin. This will help minimize any risk of contamination of the tissue system with xenogenic components, thereby making the tissue systems safe for human administration.

For general techniques relating to cell culture and culturing ES cells, which can be applied to culturing USCs, the practitioner can refer to standard textbooks and reviews, for example: E. J. Robertson, “Teratocarcinomas and embryonic stem cells: A practical approach” ed., IRL Press Ltd. 1987; Hu and Aunins (1997), Curr. Opin. Biotechnol. 8:148-153; Kitano (1991), Biotechnology 17:73-106; Spier (1991), Curr. Opin. Biotechnol. 2:375-79; Birch and Arathoon (1990), Bioprocess Technol. 10:251-70; Xu et al. (2001), Nat. Biotechnol. 19(10):971-4; and Lebkowski et al. (2001) Cancer J. 7 Suppl. 2:S83-93, each incorporated herein by reference.

The limbal cells comprising USCs are cultured or passaged in an appropriate medium to allow the USCs to remain in a substantially undifferentiated state. Although colonies of USCs within the population may be adjacent to neighboring cells that are differentiated, the culture of USCs will nevertheless remain substantially undifferentiated when the population is cultured or passaged under appropriate conditions, and individual USCs constitute a substantial proportion of the cell population. Undifferentiated stem cell cultures that are substantially undifferentiated contain at least about 20% undifferentiated USCs, and may contain at least about 40%, 60%, 80%, or 90% USCs. For example, USCs in culture must be kept at an appropriate cell density and subcultured while frequently exchanging the culture medium to prevent them from differentiating. In long term culture, when the cells are passaged they may be dispersed into small clusters or into single-cell suspensions. Typically, a single cell suspension of cells is achieved and then seeded onto another tissue culture grade plastic dish.

The cultures can be serially passaged for at least 20, 40, 60, 80, 100 or more passages, without USCs substantially differentiating. The limbal cell cultures comprising USCs can be cryopreserved for further use at various time points without loss of differential potential, preferably in freezing medium that comprises culture medium with 10-90% heat inactivated serum collected from human cord blood and 5-10% DMSO. Preferably the limbal cell cultures comprising USCs are preserved after every passage, for example by cryopreserving, so that additional or multiple tissue systems can be generated from a single limbal tissue biopsy. These cryopreserved cultures will also serve as a pool of undifferentiated, self-regenerating, and viable limbal stem cells for future use at any given point in time. For example, these cryopreserved cultures may be used to generate additional tissue systems for autologous use in the event of a failure of the tissue system in the recipient due to immunosuppression, complications from previous surgeries, infection, and the like. These cryopreserved cultures may also be used to generate additional tissue systems for biocompatible patients. The availability of these preserved cultures will also obviate the need to remove additional limbal tissue from a donor in the event the tissue system fails, thereby preventing the risk of exhausting a source of autologous limbal stem cells in the future.

The cells in the tissue system disclosed herein may be screened for the presence of USCs by screening for expression of certain stem cell specific markers, such as SSEA-4, SSEA-3, OCT-4, Nanog, TDGF, UTX-1, FGF-4, Tra-1-60, Tra-1-81, stem cell factor (SCF), Sox 2, Rex 1, as well as other gene marker of undifferentiated cells, or combinations thereof. The positive expression of these stem cell-specific markers, particularly ES cell-specific markers, indicates that the tissue system comprises USCs, which exhibit ES cell-like characteristics and properties. The cells of the tissue system may also be characterized for the presence of keratinocytes, for example by screening for the expression of specific markers which demonstrate the corneal nature of the cells in the tissue system, such as p63, K3, K12, K19, and the like, or combinations thereof. The morphology and phenotype of the tissue system may be analyzed, for example by immunofluorescence or immunoperoxidase assays. The tissue systems disclosed herein may also be screened for other markers to determine the level of differentiation, if any, necessary for the tissue systems to be used as successful transplants, implants, or grafts for repairing ocular damage.

After the tissue system disclosed herein is generated, it must be transported to the recipient's location for transplant, implant, or graft. Preferably, the means used to transport the tissue system maintains the viability of the tissue system sufficiently that it is still useful as a transplant, implant, or graft after transport. In a preferred embodiment, the tissue system is transported in a specially designed receptacle that contains transportation medium, which is preferably the enriched medium used to culture the tissue system comprising USCs. The specially designed transportation receptacle preferably comprises a portable, cylindrical housing base open at the upper end to receive the tissue system and closed at the bottom, with parallel means positioned within the upper end of the housing base for supporting the tissue system fastened on the culture insert, and a cap for closing the open upper end of the housing base. The transportation receptacle may be constructed from special tissue culture grade plastic-1, medical grade stainless steel, e.g., SS 316 L, or any other suitable grade, medical grade silicone, or any other suitable tissue culture grade material. Preferably, the receptacle for transportation holds the tissue system in a secure fashion, thereby minimizing the chances that the tissue system will be damaged during transport.

The tissue system comprising USCs disclosed herein can be utilized for therapeutic applications, for example as transplants, implants, or grafts for subjects with limbal stem cell deficiencies in one or both eyes. The tissue system of the present disclosure can be used to treat any subject in need of treatment, including but not limited to humans, primates, and domestic, farm, pet, or sports animals, such as dogs, horses, cats, sheep, pigs, cattle, rats, mice, and the like. As used herein, the terms “therapeutic”, “therapeutically”, “to treat”, “treatment”, or “therapy” refer to both therapeutic treatment and prophylactic or preventative measures. Therapeutic treatment includes but is not limited to reducing or eliminating the symptoms of a particular disease, condition, injury or disorder, or slowing or attenuating the progression of, or curing an existing disease or disorder. Subjects in need of such therapy will be treated by a therapeutically effective amount of the tissue system to restore or regenerate function. As used herein, a “therapeutically effective amount” of the tissue system is an amount sufficient to arrest or ameliorate the physiological effects in a subject caused by the loss, damage, malfunction, or degeneration of limbal stem cells. The therapeutically effective amount of cells or tissues used will depend on the needs of the subject, the subject's age, physiological condition and health, the desired therapeutic effect, the size of the area of tissue that is to be targeted for therapy, the site of implantation, the extent of pathology, the chosen route of delivery, and the treatment strategy. These tissue system is preferably administered to the patient in a manner that permits the tissue system to graft to the intended site and reconstitute or regenerate the functionally deficient area.

In preferred embodiments, the tissue system of the present disclosure is used to therapeutically treat subjects with ocular damage or disease, particular ocular surface impairments. Alternatively, the disclosed tissue system may be used to treat other diseases or damage which will therapeutically benefit from a source of undifferentiated stem cells derived from limbal tissue, for example to repair burned skin areas. The disclosed tissue system is particularly well suited to treat subjects with primary limbal stem cell deficiencies, which may be caused by hereditary conditions such as aniridia, multiple-endocrine-deficiency-associated keratitis, limbitis, and idiopathy. or secondary limbal stem cell loss, which may occur from acquired conditions such as Steven-Johnson syndrome, infections (such as severe microbial keratitis), ocular surface tumors, traumatic destruction of limbal stem cells caused by chemical or thermal injury or exposure to ultraviolet radiation, multiple surgeries or cryotherapies, corneal intraepithelial neoplasia, peripheral ulcerative or inflammatory keratitis, ischemic keratitis, keratopathy, toxic effects induced by contact lens or lens cleaning fluids, immunological conditions, ocular cicatrical pemphigoid, pterygium, pseudopterygium, and the like Preferably the tissue system is transplanted, implanted, or grafted to the subject, and is able to repair ocular damage or disease in the subject, preferably by providing a stable limbal stem cell population to the subject's damaged or diseased eye. In preferred embodiments, transplantation, implantation, or grafting of the tissue system facilitates epithelization, maintains normal epithelial phenotype, reduces inflammation, reduces scarring, reduces adhesion of tissue, reduces vascularization, and improves vision in the eye.

For example, the tissue system can be transplanted, implanted, or grafted to repair a damaged cornea. While many such methods are well known to those of skill in the art, one such method involves periotomy at the limbus, followed by removal of the perilimbal subconjunctival scar and inflamed tissues to the bare sclera. The fibrovascular tissue of the cornea may be removed by lamellar keratectomy. The tissue system can be scaled according to the size of the recipient eye, and transplanted or grafted to the corresponding recipient limbal area. Alternatively, the tissue system may be used as a whole lamellar corneal tissue, and transplanted or grafted as lamellar keratoplasty to cover the entire area. The transplanted, implanted, or grafted tissue system is then secured to the damaged site, for example with sutures or any other means known to those of skill in the art.

The following examples are included to demonstrate preferred embodiments of the disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

1) Collection of Limbal Tissue Biopsies

Prior to initiating the collection of limbal tissue biopsies from human patients, Institutional Review Board approval was obtained. Informed consent was obtained from each patient and donor, and all human subjects were treated according to the Helsinki Accord. A 0.8 mm²to 2 mm²limbal biopsy was surgically removed from the donor eye from superior or temporal quadrants of the corneal surface by lamellar keratectomy. These regions are particularly rich in limbal stem cells. After excision, the biopsy was immediately placed in a 2 ml transport vial filled with transport medium. The transport medium consisted of Dulbecco's Modified Eagles Medium (DMEM) and Ham's F-12 Medium (DMEM:F-12; 1:1) supplemented with 5% fetal bovine serum (FBS) or 5% human serum collected from cord blood, 0.5% dimethyl sulphoxide (DMSO), 2 ng/ml recombinant human epidermal growth factor (rhEGF), 5 μg/ml insulin, 5 μg/ml transferrin, 5 μg/ml sodium selenite, 0.5 μg/ml hydrocortisone, 0.1 nmol/l cholera toxin A, 50 μg/ml gentamycin, and 1.25 μg/ml amphotericin B. Blood samples were also collected from each donor and transported along with each limbal tissue biopsy to a centrally located cGMP facility. Blood samples were immediately tested for infectious diseases, including Hepatitis B virus (HBV), Hepatitis C virus (HCV), Syphillis, and CMV.

2) Culturing of Limbal Biopsies to Produce Limbal Stem Cell Cultures

Limbal tissue from the limbal biopsies was initially washed several times with ringer solution (PBS with penicillin, streptomycin, and gentamycin) and cut into small pieces. These small pieces of limbal tissue were dry incubated for 2-5 minutes, and then placed on the biocoated amniotic membrane in the insert in a circular fashion. A small amount of DMEM medium with 10% knock-out serum (150-200 μl) was added to each plate, facilitating the biopsy pieces sticking to the biocoated tissue culture surface. The next day, 2 ml of the same medium was added to the plate and incubated for 4-5 days at 37° C. in a CO₂ incubator. After 4-5 days the limbal stem cells in the culture began proliferating. At this point, the biopsies were gently and carefully removed from the culture using sterile forceps, thus allowing the limbal stem cells remaining in the culture to continue to proliferate. This method avoided the growth of mesenchymal or fibroblast cells in the culture. The limbal stem cells were allowed to grow until they reached the confluence of about 80%, typically after 7 to 21 days in culture.

3) Isolation of Undifferentiated Stem Cells from Cultured Limbal Stem Cells and Generation of Tissue Systems

After the limbal cell culture reached the desired level of confluency, the cultured cells were subjected to magnetic affinity cell sorting (MACS) to isolate pluripotent limbal stem cells that are USCs. The cultured cells were first dispersed using 0.05% trypsin-EDTA. The trypsin was neutralized by adding an equal amount of culture medium that contained a trypsin inhibitor or fetal calf serum. The cells were subsequently pipeted into a single cell suspension, and counted using a hemocytometer. Next, the cells were spun down and resuspended to a concentration of 10⁷ cells per 200 μl of phosphate buffered saline (PBS). The cells were incubated for 30 minutes at 4° C. with 1 μl of primary antibody SSEA-4 (1:60, Chemicon). After incubation with SSEA-4 primary antibody, the cells were washed twice with PBS to remove any unbound antibody. A 20 μl suspension of goat anti-mouse fluorescein isothiocyanate (FITC)-conjugate secondary antibody beads (1:10, Miltenyi Biotec), which bind to the SSEA-4 primary antibody, were added to 200 μl of the cell suspension, mixed well, and incubated at 4° C. for 20 minutes. The cells were washed three times with PBS to remove any unbound secondary antibody.

The cell suspension was then passed through a MACS magnetic column according to the manufacturer's instructions (Miltenyi Biotec) to isolate SSEA-4 positive cells. The negative fraction was collected first, and the column was washed twice with PBS. Next, the column was removed from the magnet and the positive fraction with SSEA-4 positive cells was collected.

4) Preparation of Amniotic Membrane as a Tissue Base for Tissue System

Although several suitable extracellular matrix carriers are available to serve as a tissue base for culturing limbal stem cells, such as Matrigel™, fibrinogen, PDGF, laminin, EGF, or collagen V, human amniotic membrane was used to culture limbal stem cells. The preparation of these amniotic membrane culture began with the collection of human placental membranes. Placental membranes were collected from elective Cesarean section operations and transported to laboratory facilities in a transport medium consisting of Dulbecco's phosphate buffered saline (DPBS) supplemented with 50 unit/ml penicillin, 50 unit/ml streptomycin, 100 μg/ml neomycin, and 2.5 μg/ml amphotericin B. Placental membrane was transported to the laboratory within 3 hours of surgery. Blood samples were also collected from each donor and sent for infectious disease diagnostic tests as described above.

Once received, the placenta was washed with washing medium to remove mucus and blood clots. The washing medium consisted of DPBS supplemented with 50 unit/ml penicillin, 50 unit/ml streptomycin, 100 μg/ml neomycin, and 2.5 μg/ml amphotericin B. Placental tissue was removed from the amniotic membrane using sterile scissors, and the amniotic membrane was washed thoroughly at least 7 times to remove substantially all blood clots. Next, the chorion was peeled off of the amniotic membrane with blunt forceps, and the epithelial side of the amniotic membrane was washed 5 times with the washing medium. The amniotic membrane was then placed on a sterile nitrocellulose membrane with the epithelial side of the membrane facing up. The membrane was cut into 5 cm×5 cm area pieces and each piece was placed in a cryo-vial filled with freezing medium consisting of 50% glycerol in DMEM. Each batch of processed amniotic membrane was checked for sterility, as well as the absence of mycoplasma or endotoxin contamination before being use. The pieces of amniotic membrane were each stored at −80° C.

Amniotic membrane cultures for culturing limbal stem cells were prepared from these pieces of amniotic membrane by first thawing the pieces at room temperature for 20 minutes. Each amniotic membrane was then carefully removed from the nitrocellulose membrane using blunt forceps, preferably without tearing the surface, and placed on a sterile glass slide in a 100 mm petri plate. Next, a small volume of 0.25% trypsin (1.0-1.5 ml) was added to cover the amniotic membrane, and the membrane was incubated at 37° C. for 30 minutes. After incubation, the epithelial layer of the amniotic membrane was scraped off with a cell scraper under sterile aseptic conditions. The amniotic membrane was then washed 3 times with washing solution. The processed and treated amniotic membrane, which functions as an extracellular carrier matrix or tissue base in culture, was placed on a culture plate with a 0.4 μM track-etched polyethylene terephthalate (PET) membrane insert (Millipore). The amniotic membrane was fastened to the PET insert, for example by using number 10 Ethilon non-absorbent suture or by using a medical grade silicon O-ring.

Regardless of the means, the amniotic membrane should be spread on the membrane insert in such a way that the denuded epithelial side of the membrane faces the inner side of the insert and the stromal side of the membrane faces out of the insert. The amniotic membrane was stretched uniformly before being secured to the insert, for example by inserting the silicon O-ring into the bottom of the amniotic membrane, or suturing the amniotic membrane to the basement membrane of the insert. The entire set-up was incubated in a 6-well dish filled with culture medium for at least 2 hours. After this time period, the culture medium was removed and the amniotic membrane was pre-coated with laminin, fibrinogen, or collagen IV, alone or in combination. The amniotic membrane was washed two times with culture medium and again incubated in culture medium for 30 minutes, after which the amniotic membrane was ready for culturing limbal stem cells.

5) Culturing Isolated Limbal Stem Cells Comprising USCs and Generation of Tissue System

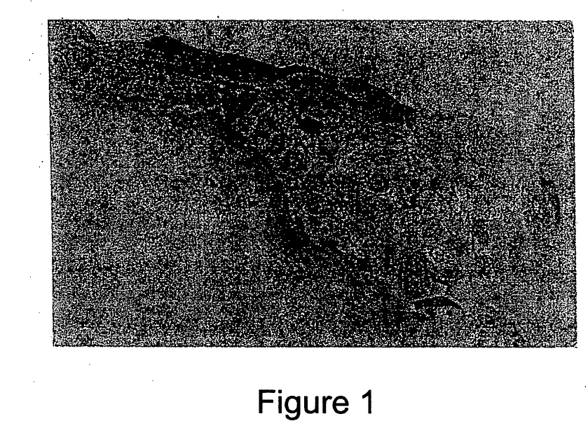

The SSEA-4 positive cells, which contain USCs, were washed twice and seeded on human amniotic membrane tissue base in culture medium. The amniotic membrane was biocoated with laminin to facilitate adherence of USCs onto the amniotic membrane in the presence of the enriched culture medium. Preferably, the culture medium was DMEM and F-12 (DMEM:F-12; 1:1), enriched with 30% conditioned medium obtained from 2 day old inactivated human embryonic fibroblasts. The culture medium was preferably further supplemented with 10% knock-out serum or 10% heat-inactivated human serum collected from cord blood, DMSO (0.5%), rhEGF (2 ng/ml), insulin (5 μg/ml), transferrin (5 μg/ml), sodium selenite (5 μg/ml), hydrocortisone (0.5 μg/ml), gentamycin (50 μg/ml), and amphotericin B (1.25 μg/ml). The USCs were cultured in this medium for a period of 10-15 days at 37° C. in a CO₂ incubator until a multi-layered tissue system with a large population of USCs was obtained. FIG. 1 shows Hematoxylin and Eosin (H & E) staining of the multi-layered tissue system on human amniotic membrane at the termination point in culture. Hematoxylin stains negatively charged nucleic acids such as nuclei and ribosomes blue, while Eosin stains proteins pink. After 10-15 days in culture the tissue system was ready for transplantation.

Alternatively, after the cell suspension was passed through a MACS magnetic column as described above to isolate SSEA-4 positive cells, the cells were seeded on Matrigel™-coated plates with culture medium. The culture medium was DMEM and F-12 (DMEM:F-12; 1:1), supplemented with 10% knock-out serum or 10% heat-inactivated human serum collected from cord blood, DMSO (0.5%), rhEGF (2 ng/ml), insulin (5 μg/ml), transferrin (5 μg/ml), sodium selenite (5 μg/ml), hydrocortisone (0.5 μg/ml), bFGF (4 ηg/ml), hLIF (10 μg/ml), gentamycin (50 μg/ml) and amphotericin B (1.25 μg/ml). The isolated cells were cultured for 8-10 days at 37° C. in a CO₂ incubator or until a multi-layered tissue system with a large population of USCs was obtained. The tissue culture was subsequently ready for transplantation.

After the sorted and isolated cells were grown in culture to confluence, certain cultures of USCs were dissociated and re-plated on fresh bio-coated tissue culture dishes at a ratio of 1:3. The USCs were then expanded and serially passaged for at least 40 population doublings, or approximately 13 passages.

### EXAMPLE 2

Analysis and Characterization of the Tissue System Containing Undifferentiated Stem Cells

As outlined in Example 1, a tissue system comprising USCs was derived from limbal tissue biopsies. To better understand the cell population of the tissue system derived from limbal tissue, cells in the tissue system were analyzed using flow cytometry, immunofluorescence and immunoperoxidase assays, and molecular analysis for the presence or absence of various cellular markers of undifferentiated cells.

1) Flow Cytometry Analysis

The cell population of the limbal biopsy cultures was compared to the cell population in the tissue system using flow cytometry analysis to detect the presence of the SSEA-4 marker. First, cells were collected from the limbal biopsy cultures at a semi-confluent stage and from the tissue system grown on an amniotic membrane tissue base after sorting and selecting cells by MACS as set forth above. The two populations of cells were analyzed separately. The collected cell populations were places in sterile PBS and resuspended into a cell suspension. Next, 100 μl aliquots of cells were permeabilized with Triton X-100 0.2% for nuclear antigens. The cells were then divided into three groups. The first group of cells were not labeled with an antibody as a control. The second group of cells were incubated with SSEA-4 primary antibody (Chemicon, 1:60) for 20 minutes at 4° C. The third group was not incubated with SSEA-4 primary antibody. Next, cells in the second and third group were washed with PBS and incubated with anti-mouse FITC-conjugate secondary antibody (Sigma, 1:500) for 20 minutes at 4° C in the dark.

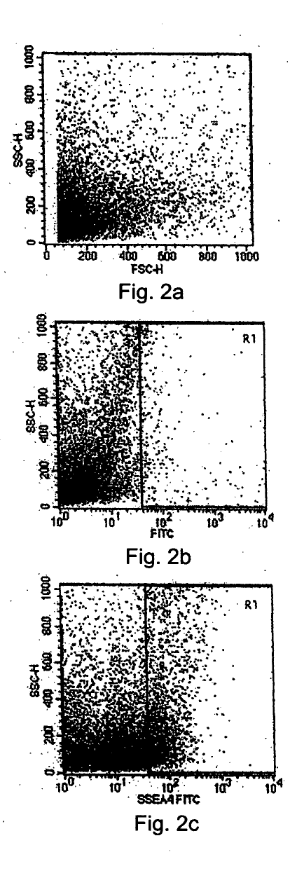

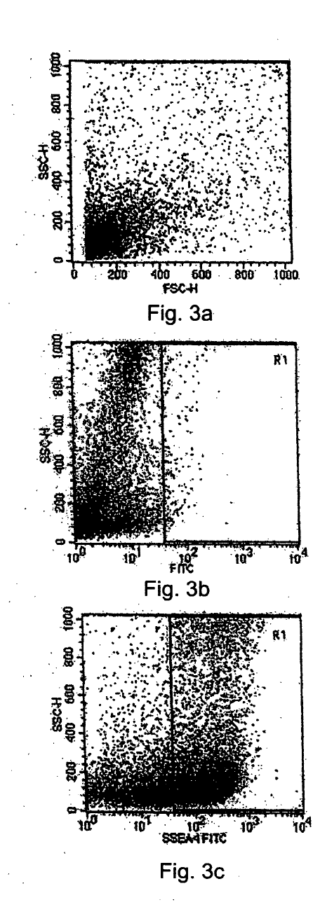

After incubation with the secondary antibody, the cells of all three groups were washed with PBS, resuspended in 400-500 μl of PBS, and loaded into a FACS Calibur flow cytometer (Becton-Dickinson). The cells were identified by light scatter, and logarithmic fluorescence was evaluated based on 10,000 gated events, with control samples used to adjust for background fluorescence. Analysis was performed using CELL QUEST software (Becton Dickinson). The results of the flow cytometry analysis for limbal biopsy cells is shown in FIG. 2, while FIG. 3 shows the same analysis for cells in the tissue system comprising USCs. FIG. 2 shows that only about 30% of the cells in the cultured limbal tissue biopsy are SSEA-4 positive cells. In contrast, FIG. 3 shows the population of SSEA-4 positive cells has been enriched to 74% of the cells present in the tissue system, indicating the high percentage of USCs present in the tissue system.

2) Immunofluorescence and Immunoperoxidase Assays

The morphology and phenotype of the tissue system was analyzed by immunofluorescence and immunoperoxidase assays. First, sections of the deparaffinized tissue system were rinsed with PBS and permeabilised with 0.2% triton X-100 in PBS, blocked with 1% bovine serum albumin/PBS, and incubated the following primary antibodies (antibody dilution was made in 1% BSA/PBS) for 2 hours at room temperature: SSEA-4 (1:60, Chemicon), Stem Cell Factor (1:250, Santacruz), Tra-1-60 (1:40, Chemicon), Oct-4 (1:100, Chemicon), Connexin43 (1:200, Chemicon), p63 (1:150, US Biologicals), K3/K12 (1:200, ICN), and K19 (1:100, Cymbus Biotechnology). The sections were subsequently incubated with FITC-labeled secondary antibody for one hour at room temperature (Sigma). After incubation, the sections were mounted in immunoflour mounting medium and photographed using a fluorescence microscope (Nikon). For the immunoperoxidase assays, the manufacturer's protocol in the Vector Elite kit was followed. The chromogen used with the immunoperoxidase assay was diaminobenzidine tetrahydrochloride.

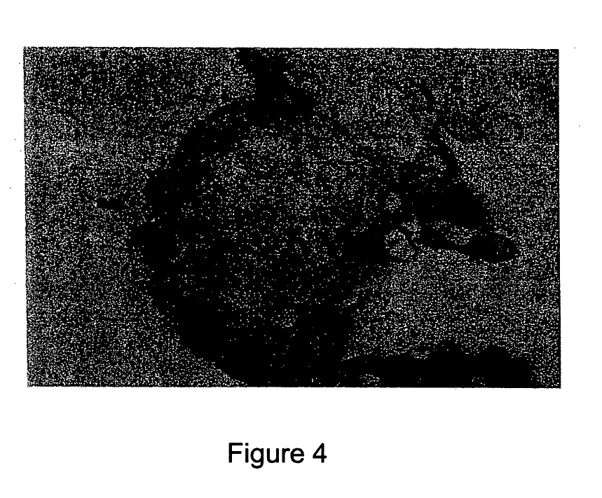

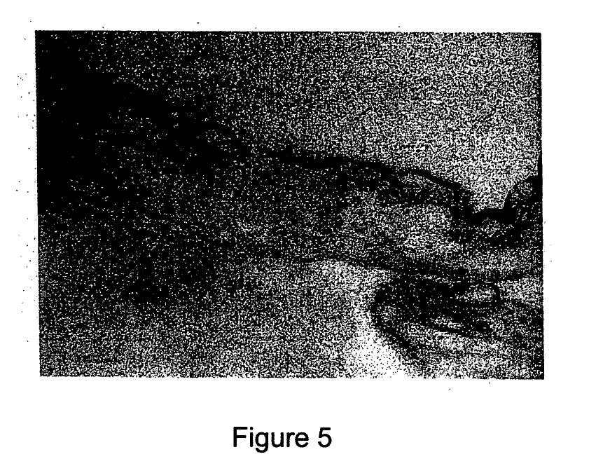

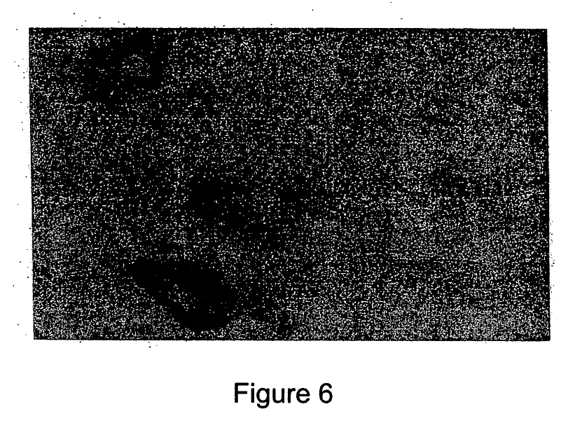

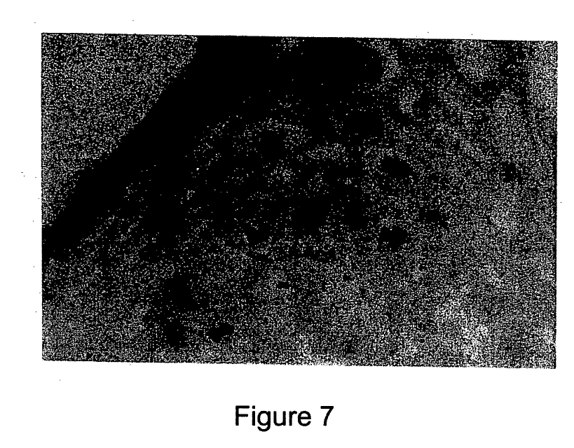

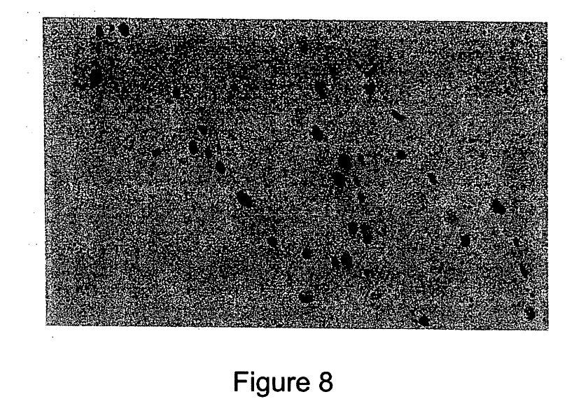

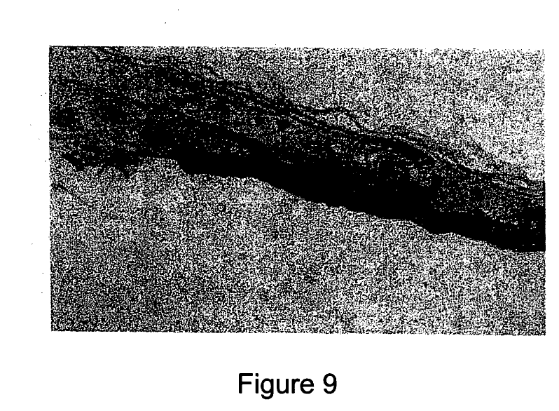

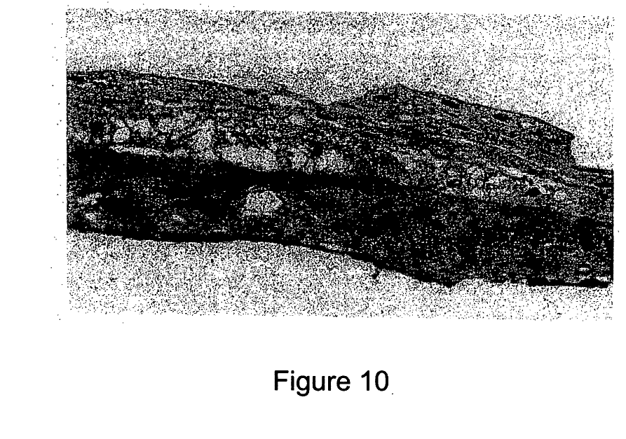

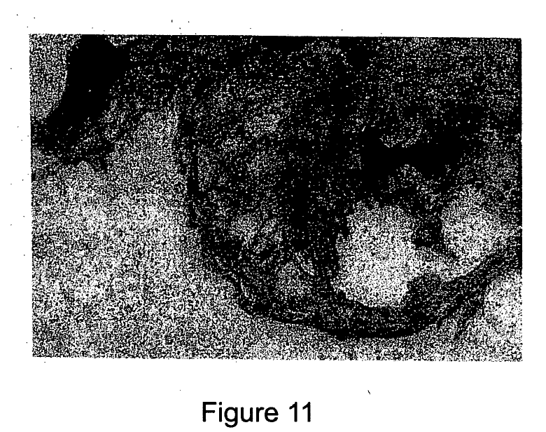

The immunofluorescence and immunoperoxidase analysis of the sections of the multi-layered tissue systems revealed the presence of about 30-70% SSEA-4 positive cells (FIG. 4), about 25-45% SCF positive cells (FIG. 5), about 30-40% Tra-1-60 positive cells (FIG. 6), about 45-55% Oct-4 positive cells (FIG. 7), and about 50-70% p63 positive cells (FIG. 8). The positive presence of K3/K12 (FIG. 9) was also detected in the tissue system, as well as basal layer expression of K19 (FIG. 10). Analysis of the tissue system sections also revealed the absence of connexin 43 (FIG. 11). The presence of stem cell specific surface markers in the sections of the tissue system confirms the presence of USCs with self-regenerating capacity in the tissue system. Other markers, namely K3/K12, K19, and p63, indicate the corneal nature of the limbal stem cells in the tissue system, which is important for successful use of the tissue system for ocular repair.

3) Molecular Analysis

To further characterize the USCs present in the tissue system, the cells were analyzed by RT-PCR for expression of the following undifferentiated stem cell marker genes: Oct-4, Nanog, Rex1, bone morphogenic protein 2 (BMP2), and bone morphogenic protein 5 (BMP5). Expression of Oct-4, Nanog, and Rex1 are down regulated upon differentiation. Expression of BMPs indicates that cells are of ectodermal origin. Expression of the “housekeeping” gene GAPDH, which is ubiquitously expressed in all cells, was also analyzed as a positive control. The identity of the RT-PCR products was confirmed by sequencing.

Briefly, total RNA of cells isolated from the tissue system generated in Example 1 was isolated using the TRIzol method (Gibco-BRL). Next, 1 μg of total RNA treated with RNase-OUT ribonuclease inhibitor (Invitrogen Inc, USA) was used for cDNA synthesis by reverse-transcription using Moloney Murine Leukemia Virus Superscript II and oligo dT (Invitrogen Inc, USA) to prime the reaction. For each polymerase chain reaction (PCR) reaction, 20 μl of cDNA was amplified by PCR using Abgene 2X PCR master mix and the appropriate primers. PCR primers were selected to distinguish between cDNA and genomic DNA by using individual primers specific for different exons. The primers used to amplify Oct-4, Nanog, Rex1, BMP2, BMP5, and GAPDH cDNAs (Genosys) are set forth below in Table 1. The PCR amplification conditions used in the thermal cycler (ABI Biosystems 9700) to amplify the PCR products were 94° C. for 30 seconds; annealing Tm ° C. (52-65° C.) for 1 minute, and 72° C. for 1 minute, for 30-35 cycles.

TABLE 1

| | | | PCR |
|---|---|---|---|
| | | Annealing | Product |
| Gene | Primer sequence | Temp (° C.) | size (bp) |
| GAPDH | 5′-TGAAGGTCGGAGTCAACGGATTTGGT-3′ | 60 | 890 |
| | (SEQ ID NO:1) | | |
| | 5′-CATGTGGGCCATGAGGTCCACCAC-3′ | | |
| | (SEQ ID NO:2) | | |
| Oct-4 | 5′-CGRGAAGCTGGAGAAGGAGAAGCTG-3′ | 58 | 247 |
| | (SEQ ID NO:3) | | |
| | 5′-CAAGGGCCGCAGCTTACACATGTTC-3′ | | |
| | (SEQ ID NO:4) | | |
| Nanog | 5′-CCTCCTCCATGGATCTGCTTATTCA-3′ | 52 | 262 |
| | (SEQ ID NO:5) | | |
| | 5′-CAGGTCTTCACCTGTTTGTAGCTGAG-3′ | | |
| | (SEQ ID NO:6) | | |
| Rex1 | 5′-GCGTACGCAAATTAAAGTCCAGA-3′ | 56 | 306 |
| | (SEQ ID NO:7) | | |
| | 5′-CAGCATCCTAAACAGCTCGCAGAAT-3′ | | |
| | (SEQ ID NO:8) | | |
| BMP2 | 5′-GGAAGAACTACCAGAAACGCG-3′ | 55 | 657 |
| | (SEQ ID NO:9) | | |
| | 5′-AGATGATCAGCCAGAGGAAAA-3′ | | |
| | (SEQ ID NO:10) | | |
| BMP5 | 5′-AAGAGGACAAGAAGGACTAAAAATAT-3′ | 55 | 303 |
| | (SEQ ID NO:11) | | |
| | 5′-GTAGAGATCCAGCATAAAGAGAGGT-3′ | | |
| | (SEQ ID NO:12) | | |

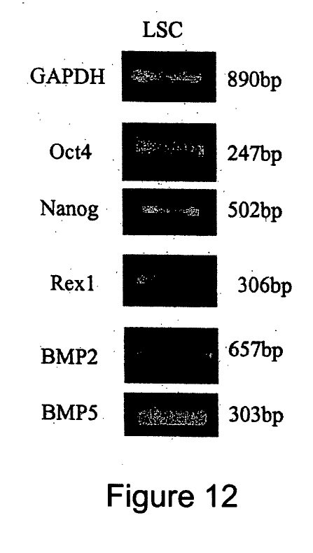

The results of this experiment are shown in FIG. 12, and demonstrate expression of all genes tested. Expression of Oct-4, Nanog, and Rex1 markers indicates that USCs present in the tissue system are undifferentiated.

### EXAMPLE 3

Viability Studies of the Tissue System Comprising Undifferentiated Stem Cells:

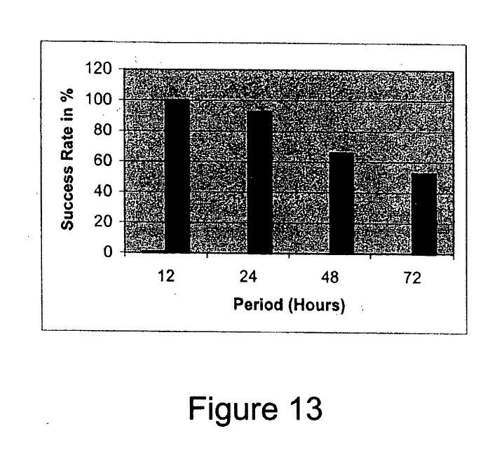

Limbal tissue biopsies were evaluated to determine how long the biopsies could remain in transport prior to in vitro culturing of a viable tissue system with USCs. The limbal tissue biopsies were transported or stored in the following culture media for 12, 24, 48, and 72 hours after surgery at 4° C.: DMEM and Ham's F-12 (ratio 1:1), supplemented with human cord blood serum (3-5%), DMSO (0.5%), rhEGF (2 ng/ml), insulin (5 μg/ml), transferrin (5 μg/m), sodium selenite (5 μg/ml), hydrocortisone (0.5 μg/ml), cholera toxin A (0.1 nmol/l ), gentamycin (50 μg/ml), and amphotericin B (1.25 μg/ml). After this period of time, the biopsies were cultured to generated tissue systems with USCs as described in Example 1. FIG. 13 shows the percent success in developing a tissue system from biopsies cultured approximately 12, 24, 48 and 72 hours after surgical collection from a subject. As shown in FIG. 13, the explants are preferably cultured within about 24 hours after a biopsy is collected, with the greatest success for generating tissue systems achieved with biopsies cultured within about 12 hours of collection. However, the biopsies retained significant ability to generate tissue systems in culture even up to about 72 hours after surgical collection.

The viability of the tissue system generated in Example 1 during transportation was evaluated to determine how long after removal from culture the cultured tissue system would remain viable for use as a tissue transplant. The tissue system was placed in a specially designed receptacle containing transportation medium, and transported at room temperature or at 4° C. The transportation medium consisted of DMEM and F-12 (DMEM:F-12; 1:1), enriched with 30% conditioned medium obtained from 2 day old inactivated human embryonic fibroblasts, and further supplemented with 10% knock-out serum or 10% heat-inactivated human serum collected from cord blood, DMSO (0.5%), rhEGF (2 ng/ml), insulin (5 μg/ml), transferrin (5 μg/ml), sodium selenite (5 μg/ml), hydrocortisone (0.5 μg/ml), gentamycin (50 μg/ml), and amphotericin B (1.25 μg/ml). The viability of the tissue system was checked at intervals of 6, 12, 24, and 48 hours. The viability of the tissue system in transportation medium was assessed based on parameters such as pH of the medium, viability of the cells, percentage of dead cells, and integrity of the tissue system architecture (e.g., evaluated via flat mount).

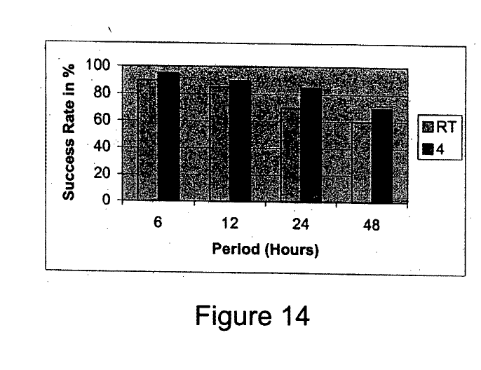

FIG. 14 shows the viability of the tissue system at various time periods post-culture depending on the temperature at which the tissue system was transported. Slightly better results were obtained when the tissue system was transported at 4° C. than when it was transported at room temperature, and retained excellent viability for the first 12 hours of transportation, with good viability still found after 48 hours.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A tissue system comprising limbal stem cells, wherein at least about 30-90% of the limbal stem cells in the tissue system are undifferentiated stem cells.

2. The tissue system of claim 1, wherein the undifferentiated stem cells express one or more stem cell specific markers selected from the group consisting of SSEA-4, SSEA-3, Oct-4, Nanog, Rex-1, Stem Cell Factor, Tra-1-60, CD73, CD105, CD31, CD54, and CD117.

3. The tissue system of claim 1, wherein the undifferentiated stem cells express SSEA-4.

4. The tissue system of claim 3, wherein the undifferentiated stem cells expressing SSEA-4 comprise at least about 50-90% of the limbal stem cells in tissue system.

5. The tissue system of claim 1, wherein the limbal stem cells express one or more cell surface markers selected from the group consisting of K3/K12, K19, and p63.

6. The tissue system of claim 1, wherein the tissue system is derived from comeoscleral limbus tissue.

7. The tissue system of claim 6, wherein the comeoscleral limbus tissue is human tissue.

8. The tissue system of claim 1, wherein the tissue system is a multi-layered tissue system.

9. The tissue system of claim 1, wherein the tissue system is suitable for transplantation, implantation, or grafting to a recipient.

10. A method of generating a tissue system, wherein the tissue system comprises limbal stem cells, comprising the steps of:
(a) isolating corneal limbal tissue from a donor;
(b) culturing the corneal limbal tissue to expand corneal limbal cells in culture;
(c) isolating a population of limbal stem cells from the cultured corneal limbal cells by sorting the corneal limbal cells to select for one or more stem cell-specific surface markers, wherein the stem cell-specific surface marker is expressed by undifferentiated stem cells;
(d) culturing the isolated population of limbal stem cells to generate the tissue system.

11. The method of claim 10, wherein the limbal stem cells comprise undifferentiated stem cells.

12. The method of claim 11, wherein the undifferentiated stem cells comprise at least about 70% of the limbal stem cells in the tissue system.

13. The method of claim 11, wherein the undifferentiated stem cells express one or more stem cell specific markers selected from the group consisting of SSEA-4, SSEA-3, Oct-4, Nanog, Rex-1, Stem Cell Factor, Tra-1-60, CD73, CD105, CD31, CD54, and CD117.

14. The method of claim 13, wherein the undifferentiated stem cells express SSEA-4.

15. The method of claim 14, wherein the undifferentiated stem cells expressing SSEA-4 comprise at least about 50-90% of the limbal stem cells in the tissue system.

16. The method of claim 10, wherein the undifferentiated stem cells express one or more cell surface markers selected from the group consisting of K3/K12, K19, and p63.

17. The method of claim 10, wherein the corneal limbus tissue is isolated from a human donor.

18. The method of claim 10, wherein the tissue system is suitable for transplantation, implantation, or grafting to a recipient.

19. The method of claim 18, wherein the recipient has a limbal stem cell deficiency in one or both eyes.

20. The method of claim 18, wherein the donor is the same as the recipient.

21. The method of claim 18, wherein the donor is biocompatible with the recipient.

22. The method of claim 10, wherein the corneal limbal tissue is cultured on a biocoated surface or an extracellular matrix carrier.

23. The method of claim 22, wherein the biocoated surface is a biocoated petri dish.

24. The method of claim 23, wherein petri dish is biocoated with one or more attachment factors selected from the group consisting of fibrinogen, laminin, collagen IV, tenascin, fibronectin, collagen, bovine pituitary extract, EGF, hepatocyte growth factor, keratinocyte growth factor, and hydrocortisone.

25. The method of claim 22, wherein the extracellular matrix is selected from the group consisting of Matrigel™, mammalian amniotic membrane, laminin, Reliseal™, thrombin, tenascin, entactin, hyaluron, fibrinogen, collagen-IV, poly-L-lysine, gelatin, poly-L-ornithin, fibronectin, and platelet derived growth factor (PDGF).

26. The method of claim 22, wherein the extracellular matrix is human amniotic membrane.

27. The method of claim 22, further comprising the step of dissociating the cultured corneal limbal cells prior to isolating the limbal stem cells.

28. The method of claim 10, wherein the corneal limbal tissue is cultured in culture media supplemented with one or more soluble factors selected from the group consisting of dimethyl sulphoxide, recombinant human epidermal growth factor, insulin, sodium selenite, transferrin, hydrocortisone, basic fibroblast growth factor, and leukemia inhibitory factor.

29. The method of claim 10, wherein the corneal limbal cells are sorted using magnetic affinity cell sorting (MACS).

30. The method of claim 10, wherein the corneal limbal cells are sorted using fluorescence-activated cell sorting (FACS).

31. The method of claim 10, wherein the one or more stem cell-specific surface markers for isolating the corneal limbal cells are selected from the group consisting of SSEA-4, SSEA-3, Oct-4, Nanog, Rex-1, Stem Cell Factor, Tra-1-60, CD73, CD105, CD31, CD54, and CD117.

32. The method of claim 10, wherein the stem cell-specific surface marker for isolating the corneal limbal cells is SSEA-4.

33. The method of claim 10, wherein the isolated population of limbal stem cells are cultured on a tissue base to generate the tissue system.

34. The method of claim 33, wherein the tissue base comprises a biocoated support material.

35. The method of claim 34, wherein the biocoated support material is one or more attachment factors selected from the group consisting of fibrinogen, laminin, collagen IV, tenascin, fibronectin, collagen, bovine pituitary extract, EGF, hepatocyte growth factor, keratinocyte growth factor, and hydrocortisone.

36. The method of claim 33, wherein the tissue base is selected from the group consisting of human amniotic membrane, laminin, collagen IV, tenascin, fibrinogen, entactin, hyaluron, Reliseal™, thrombin, Matrigel™, and fibronectin.

37. The method of claim 33, wherein the tissue base is human amniotic membrane.

38. The method of claim 35, wherein the human amniotic membrane is biocoated with one or more attachment factors selected from the group consisting of laminin, collagen IV, tenascin, fibrinogen, entactin, hyaluron, Reliseal™, thrombin, Matrigel™, and fibronectin.

39. The method of claim 10, wherein the isolated population of limbal stem cells is cultured in medium enriched with conditioned medium obtained from inactivated human embryonic fibroblast cells.

40. The method of claim 10, wherein the isolated population of limbal stem cells is cultured in medium enriched with human leukemia inhibitory factor.

41. The method of claim 10, wherein the isolated population of limbal stem cells is cultured in culture media supplemented with one or more soluble factors selected from the group consisting of dimethyl sulphoxide, recombinant human epidermal growth factor, insulin, sodium selenite, transferrin, and hydrocortisone.

42. The method of claim 10, further comprising serially passaging the population of isolated limbal stem cells for at least 10, 15, or 20 passages.

43. The method of claim 10, further comprises cryopreserving the population of limbal stem cells in freezing medium.

44. The method of claim 43, wherein the freezing medium comprises culture medium with 10-90% heat inactivated human cord blood serum and 5-10% DMSO.

45. The method of claim 42, wherein the population of limbal stem cells is cryopreserved preferably after every passage.

46. The method of claim 18, further comprising transporting the tissue system to the recipient in a transportation receptacle comprising transportation medium.

47. The method of claim 46, wherein the transportation receptacle comprises a portable, cylindrical housing base open at the upper end to receive the tissue system and closed at the bottom, with parallel means positioned within the upper end of the housing base for supporting the tissue system, and a cap for closing the open upper end of the housing base.

48. The method of claim 47, wherein the transportation receptacle is constructed from special tissue culture grade plastic-1 or medical grade stainless steel.
